(19) United States
(12) Patent Application Publication
(10) Pub. No.: US 2024/0058406 A1
(43) Pub. Date: Feb. 22, 2024

(54) SYSTEM AND METHODS FOR EXTRACTION OF CANNABINOIDS AND CARBOXYLIC ACID COMPOUNDS FROM HEMP

(71) Applicant: NovelDivide, LLC, Kyle, TX (US)

(72) Inventors: Alvaro Tico Martinez, Buda, TX (US); Jong Moon Kim, McKinney, TX (US); Brian Jong Kim, Spring, TX (US)

(73) Assignee: NovelDivide, LLC, Kyle, TX (US)

(21) Appl. No.: 18/233,322

(22) Filed: Aug. 13, 2023

Related U.S. Application Data
(60) Provisional application No. 63/398,314, filed on Aug. 16, 2022.

(51) Int. Cl. A61K 36/185; A61K 31/05

(52) U.S. Cl. CPC A61K 36/185; A61K 31/05; A61K 2236/30

(57) ABSTRACT

Systems, apparatus, and methods use a basic solution in a pH range to extract non-purified cannabinoids, specifically cannabinoid acids, and carboxylic acid compounds along with their decarboxylated cannabinoid counterparts from hemp plant tissue, such as biomass or flower, using automated agitation, filtrating, and draining. The systems, apparatus, and methods agitate and cause turbulence of the hemp tissue, which allows extraction of both polar and non-polar cannabinoids in a basic solution of water as the solvent and a weak alkaline additive as the solute. The systems, apparatus, and methods extract the dominant cannabinoids and their polar cannabinoid acid counterparts without the use of continuous distillation via a catalyst, and may be performed at ambient room temperature. The extracted materials and solution are then drained from the apparatus.

### CROSS-REFERENCE TO RELATED APPLICATIONS

This application claims the benefit of U.S. Provisional Application No. 63/398,314, filed Aug. 16, 2022.

### TECHNICAL FIELD

The invention generally relates to extraction of cannabinoid and cannabinoid acid material from hemp and, more particularly, to extracting polar and non-polar cannabinoids in oxidation-reduction reactions from hemp.

### BACKGROUND OF THE INVENTION

Hemp fiber (Cannabis sativa L.), commonly known as permitted hemp tissue, does not have a Delta-9 tetrahydrocannabinol (Delta-9-THC) content that exceeds 0.3% relative to the dry plant mass. In general, it is known that the plant part of Cannabis sativa L, with the exception of the seeds, may contain cannabinoids. There are well over 128 identifiable cannabinoids, including their precursor carboxylic acids, within the Cannabis sativa L. plant. They are primarily stored within the spherical resin heads of the glandular trichomes. These cannabinoids have been known to interact with the endocannabinoid (eCB) system (ECS). For example, cannabinoid receptors, such as CB1R of the nervous system and CB2R of the immune system, have known mediated interactions with cannabinoids from Cannabis sativa L. These “specificities” in cannabinoid pharmacology and therapeutics have been studied in humans (Homo sapiens) as well as other mammals (Class: Mammalia).

Extracted cannabinoid and cannabinoid acid material are attracting more consumers where hemp derivatives are legalized due to the higher potency of their therapeutic properties. Scientific evidence has shown that compounds like Cannabis sativa L. may give relief to patients suffering from chronic pain, glaucoma, migraines, epilepsy, and provide remedial benefits for such conditions as inflammation, pain, and nausea. The non-psychoactive cannabinoids and their acids have been shown to have anti-inflammatory properties potentially useful in the treatment of symptoms of chronic pain and inflammation as well as arthritic ailments. As a consequence, sales of non-decarboxylate extracts are a continuous growing market for both the medical and recreational fields. Therefore, expanding production of extracted cannabinoid and cannabinoid acid products are important for the growth of the hemp industry.

Industries are developing new machinery and extraction systems to extract compounds from hemp material. There are three primary methods of solvent extraction, including, but not limited to, hydrocarbon extraction using either butane or propane; sub- and supercritical carbon dioxide extraction; and ethanol extraction, which is the most common methodology. Common industrial practices use dry hemp tissue with a moisture rate between 5% to 10% that consists of flower, excluding plant stem, and fan leaves or biomass including all plant tissue.

For example, prior efforts include U.S. Pat. No. 6,403,126B1, which describes a method of extracting cannabinoids, flavonoids, or essential oils in which the solvent may be hydrocarbon (e.g., toluene or trimethylpentane), a low molecular weight alcohol (e.g., ethanol), a low molecular weight chlorinated hydrocarbon (e.g., chloroform), dichloromethane, or a supercritical fluid (e.g., CO₂). It also describes extracting various products from hemp lacking Delta-9-THC obtained by passing the extract over a chromatographic column. U.S. Pat. No. 10,625,176B2 describes a method using dimethyl ether as an effective solvent to extract polar and non-polar compounds from plant material and purification of the isolated cannabinoids using distillation. U.S. Pat. Pub. No. US20160228787A1 describes extraction using numerous commercially significant plants by a method and apparatus for continuous extraction of plant oils using an advantageous azeotrope of ethanol and water and employing the different solubility of plant oils in ethanol and water to drive formation of a non-toxic oil tincture. U.S. Pat. No. 9,399,180B2 describes an extraction using a vertical process in which liquid gas is applied at the top, a vacuum draws down the extractant, and the gas is removed by cooling and recycled. U.S. Pat. No. 9,782,691B2 describes a process in which supercritical or subcritical CO₂, alone or with a lower vapor pressure gas or gases, is collected and reused in a closed-loop extraction process. U.S. Pat. No. 9,358,259B2 describes a process of extracting cannabinoids in which a strong solvent is passed through material many times in a cycle, the extractant remaining behind in a collection vessel from which the solvent is removed and recycled. U.S. Pat. Nos. 9,669,328B2, 9,789,147B2, and 9,844,740B2 describe a process of extraction that uses liquid gases after which the product is pressured through filter cartridges within a thermally insulated system.

These methods may lack the ability to extract cannabinoids and terpenes from surface trichomes of whole wet and semi-wet plant material on an industrial and commercial level in an apparatus without high cost, and/or long periods of time of saturation of hemp tissue between cycles.

Common methods to form a partially refined crude product of cannabinoids and their acid counterparts entail: (1) removing impurities from dry hemp tissue using filtration and hydrocarbons; (2) removing further impurities from the partially refined crude extraction using hydrocarbons; (3) removing impurities from the crude product to form a partially refined product using a saline water solution wash with hexane; (4) evaporating hexane from the partially refined product; (5) removing further impurities from the partially refined product using a saline water solution wash with petroleum ether; and (6) evaporating petroleum ether from the partially refined product to result in a product that comprises cannabinoids and cannabinoid acids. Additional methods use rotary evaporation and/or fractional distillation to separate solvent from mixture. Such additional distillation methods may add excessive costs, reclamation and partial loss of recycled solvent, and time for the separation and conversion of cannabinoids and cannabinoid acids from hemp tissue.

As the hemp industry rapidly grows and expands, efforts have been made to improve the extraction efficiency and overall economics of such processes. These methods of physical processing of the plant may result in increased labor intensity, time of extraction, and cost due to the use of industrial-sized apparatus, which must reaccumulate solvent for environmental conservation and safety. Hydrocarbon extraction likely would not be able to extract from biomass (homogenized) and/or freshly cut material. Ethanol extraction may require a presoak time of 8 to 24 hours while carbon dioxide forced extraction may take 8 to 10 hours total. Two economies to scale may help: (1) the ability to process hemp plant tissue having all forms of moisture content (i.e., wet, semi-wet, and dry) in its whole intact form may dramatically reduce costs for growers, processors, and consumers; and (2) apparatus capable of extracting from such material without the high cost of solvents to be re-accumulated with expensive industrial apparatus, long times, and labor expense.

### SUMMARY OF THE INVENTION

In accordance with embodiments of the present disclosure, an automatic and improved system, apparatus, and methods are described herein for extracting from hemp (Cannabis sativa L.) plant tissue both polar and non-polar cannabinoids in an oxidation-reduction reaction, primarily CBDA (cannabidiolic acid) and, optionally, CBD (cannabidiol). These embodiments may include using an automated multi-stage agitation apparatus, which may provide a primary extraction of cannabinoids, terpenes, flavonoids, and chlorophyl from hemp material. The extracted cannabinoids may include the following: cannabinoid acids, specifically, cannabidiolic acid (CBDA), cannabigerolic acid (CBGA), and/or cannabichromene acid (CBCA) in an oxidized state. The embodiments may include extracting cannabidiol (CBD) and these acids suspended in a weak alkaline solution. The embodiments may include evacuating the solution from a holding tub. The solution may be a selective weak alkaline basic solution with a potential of hydrogen (pH) of 10±2 inclusive, and the embodiments may work without needing decarboxylation or heating of the hemp plant tissue, whether it is in a wet or dry state or a combination of both.

In accordance with embodiments of the present disclosure, the agitation apparatus and methods may separate CBDA and oxidized cannabinoids within the hemp tissue (Cannabis sativa L.) without preparation of cutting, mashing, splitting, cleaving, or combining of the hemp tissue, and whether wet or dry or a combination of both, and/or in flower and/or biomass form. Within the agitation apparatus there may be multiple stages that offer dual functionality. For example, in accordance with one embodiment of the present disclosure, dual use of a stainless-steel mesh basket or drum means that the mesh basket holds hemp tissue contained in it while also allowing the cannabinoid acids and other cannabinoids to pass through it. Centrifugal force may be used to push or force cannabinoid acids and other cannabinoids out of the hemp tissue and through the mesh basket. An agitation disc with fins also may be included in the apparatus, which may be used to break down the hemp tissue to release the cannabinoid acids and other cannabinoids (e.g., bioactive molecules) from within the glandular trichomes of the hemp tissue. An outer holding tub enclosing the mesh basket and agitation disc also may be included to contain the conjugate acid-base pairs of the cannabinoid acids and cannabinoids in solution before being evacuated via a draining device.

In accordance with embodiments of the present disclosure, a partial Lewis Base reaction may be used in which the weak alkaline base will pick up a single hydrogen ion of the cannabidiolic acid (i.e., other acid forms of cannabinoids), leaving a conjugate acid-base pair of the cannabinoid. Based on the Brønsted-Lowry theory, in which the solvent is filtered water with pH 7-8 the cannabidiolic acid may be suspended within the product-favored aqueous solution (meaning there will be more available hydroxyl (OH⁻) ions, which leaves the cannabinoid acid in oxidized form) when the value of K (i.e., the equilibrium ratio of products to reactants) is greater than the concentration of reactants, and in which the measure of the OH⁻ (i.e., pOH), is in greater than equilibrium. Thus, excess OH⁻ may accept a single hydrogen (H⁺) ion from the cannabinoid acids that dissociates in equilibrium, leaving soluble cannabinoid acid ions (i.e., partially negatively charged), For this, the methods may involve first agitating hemp tissue in a mixture of a selective water-based weak alkaline solute in water solvent, allowing all and/or dominant cannabinoid acids to be removed from such tissue without acetylation in an acid catalyst.

In accordance with embodiments of the present disclosure, extraction may be performed using a specific system, including and agitation apparatus, and methods for producing the oxidized state of the following: CBDA, CBCA, and/or CBGA from hemp tissue, including by refluxing hemp tissue with water-based solvent at a temperature preferably at or near ambient room temperature, for example, in the range≥15.5° C. to ≤26.5° C. (or ≥59.9° F. to ≤79.7° F.) with the range≥21° C. to ≤26.5° C. (or ≥69.8° F. to ≤79.7° F.) being preferable to the lower range of ≥15.5° C. to <21° C. (or ≥59.9° F. to <69.8° F.). It is even more preferable for the temperature to be 21° C. (or 69.8° F.) to form an oxidized crude cannabinoid extraction. It should be understood that such extraction may be possible over the temperature range≥10° C. to ≤40.56° C. (or ≥50° F. to ≤105° F.).

The starting hemp tissue may have the following moisture characteristics: dry or cured (e.g., 10% or lower moisture content), semi-dried (e.g., >10% to 50% moisture content) or fresh hemp tissue (e.g., >50% moisture content) of either biomass (including but not exclusive to fan leaves and stems) or flower. The system, apparatus and methods preferably work with hemp tissue of moisture content in the ramie 5% to 75% inclusive.

In accordance with embodiments of the present disclosure, the agitation apparatus may provide an agitation device: having an outer tub; a mesh (e.g., stainless-steel) basket or drum contained within the tub; a motor(s), pulleys, and belt drive system; a rotational shaft(s); a disc with wings (or a propeller); a clutch(es); multiple external connectors; and a draining device. The agitation apparatus may also include a smart function switch(es) within or as part of a control panel and include the capability for wireless or wired communication (that may within or as part of the control panel) with an electronic circuit(s) of the controller in communication with or as part of the system or apparatus to control the functionality of the system, apparatus and methods disclosed herein. These and other features, aspects, and advantages of the system, apparatus, and methods will be described further with reference to the following illustrations, description, and claims.

### BRIEF DESCRIPTION OF THE DRAWINGS

The following drawings illustrate embodiments in accordance with the present disclosure, which should not be construed as being restrictive in scope in any way.

FIG. 1 is a side perspective view of an agitation apparatus in accordance with embodiments of the present disclosure.

FIG. 2 is a front-side perspective view of an outer stainless-steel tub of the agitation apparatus of FIG. 1.

FIG. 3 is a side perspective view of a detachable inner mesh basket or drum of the agitation apparatus of FIG. 1, which is reinforced with cage (e.g., stainless-steel) supports, a bottom plate (e.g., stainless-steel), and turbulence nodes, bumps, lumps, notches, indentions, humps, or the like.

FIG. 4 is a cutaway view of the agitation apparatus of FIG. 1.

FIG. 5 is a side perspective view of an agitation disc of the agitation apparatus of FIG. 1.

FIG. 6 is side view of an agitation apparatus motor, pulleys assembly, and belts of the agitation apparatus of FIG. 1.

FIG. 7 is a cross-sectional view of an agitator device of the agitation apparatus of FIG. 1, which illustrates an outer shaft that attaches to the mesh basket or drum and also illustrates an electronic clutch device of the agitator device used to engage or disengage the basket or drum from an outer shaft for rotation or to stop rotation of the basket or drum.

FIG. 8 is a side perspective view of a drain device of the agitation apparatus of FIG. 1, in which a solution containing, for example, cannabidiolic acid, cannabidiol, and all or some other minor cannabinoids, may be evacuated therefrom.

FIG. 9 is a front side perspective view of an automated control panel of the agitation apparatus of FIG. 1 with control switches and keys, an electronic circuit board for programing agitation of a mixing disc or propeller, timing of agitation, connecting and disconnecting a clutch and a shaft of the inner mesh basket or drum, an inner mesh basket or drum spin control via a controller, and a drain controller with timer.

FIG. 10 is a side perspective view of a stainless-steel hinge top cover or lid of the agitation apparatus of FIG. 1 with two separate inputs having food grade (e.g., stainless-steel) connectors for solvent and solute input.

FIG. 10A is a blowup view of a portion of a lid of the agitation device of FIGS. 1 and 10 showing a bushing or bearing that fits on an upper end of the shaft of FIG. 5.

FIGS. 11A and 11B illustrate a flowchart of the operations of the system, apparatus, and methods described herein, in accordance with embodiments of the present disclosure.

Specific details are set forth herein to provide a more thorough understanding of embodiments of the present disclosure. However, these embodiments may be practiced without all of these details or with additional details. In certain instances, well known elements may not be shown or described in detail and repetitions of steps and features may be omitted to avoid unnecessarily obscuring the embodiments. For example, various pumps, valves, jackets and lines may not be shown for clarity. Accordingly, the specification and drawings are to be regarded in an illustrative, rather than a restrictive, sense.

### DESCRIPTION

This application claims the benefit of U.S. Provisional Application No. 63/398,314, filed Aug. 16, 2022, which is incorporated herein in its entirety for all purposes.

Hereinafter, exemplary embodiments in accordance with the present disclosure will be described in detail with reference to the accompanying drawings. The drawings are not necessarily to scale and, in some instances, proportions may be exaggerated to clearly illustrate features of the various embodiments. This description is not to be taken in a limiting sense, but is made merely for the purpose of illustrating the general principles of the embodiments.

In accordance with embodiments of the present disclosure, hemp tissue extracts may be produced. The hemp tissue extracts may be in the form of a liquid and, in other embodiments, the hemp tissue extracts may be in the form of a resin. The hemp tissue extracts may include at least one cannabinoid in an organic solvent. The organic solvent may be a weak alkaline solution of pH 10±2 inclusive.

Raw hemp tissue (Cannabis sativa L.), such as flower and/or biomass may be provided or dispensed into the agitation apparatus by manual, semi-automated, or automated techniques (e.g., by hopper, crane, or any vessel holding hemp tissue (not shown)). The hemp tissue, as discussed, may be dry, semi-dry, or wet hemp flower or biomass (i.e., flower, leaf, and/or small broken stem material, such as ≤0.1 inch in width and ≤4 inches in length), excluding large main stock, stem, or long stem material that may contain no or little cannabinoids

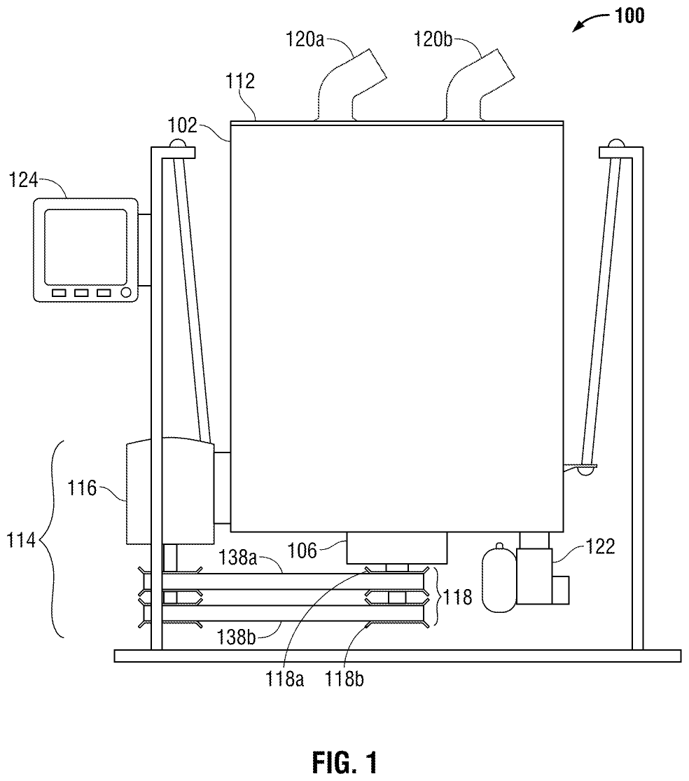

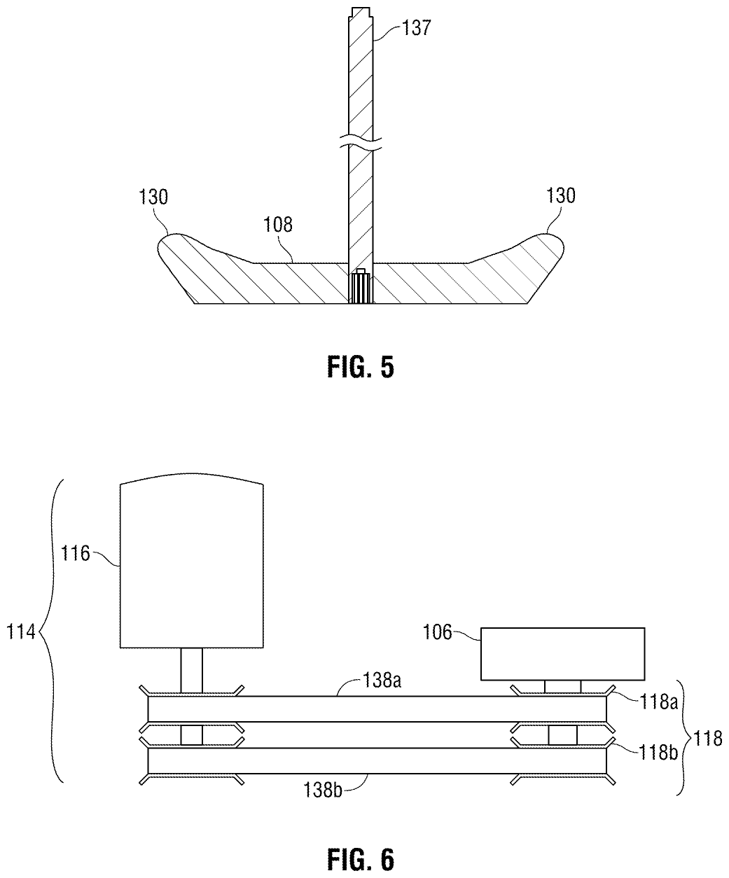

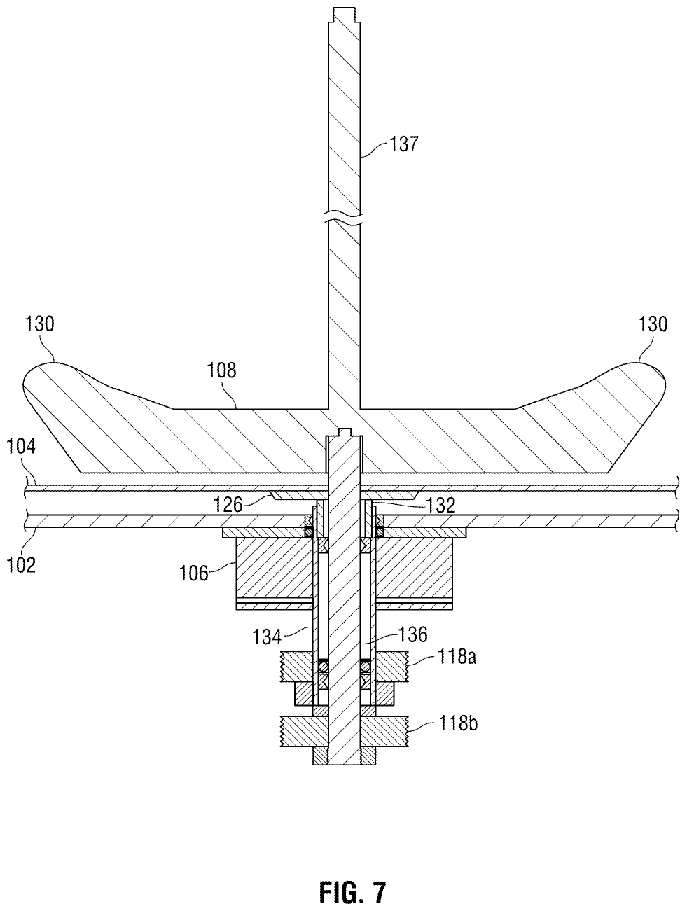

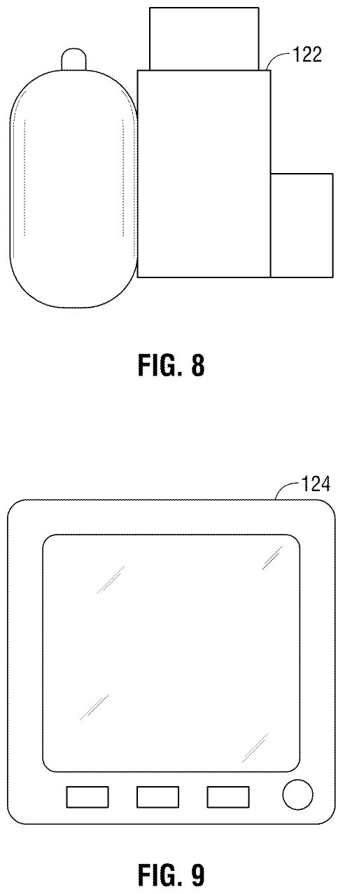

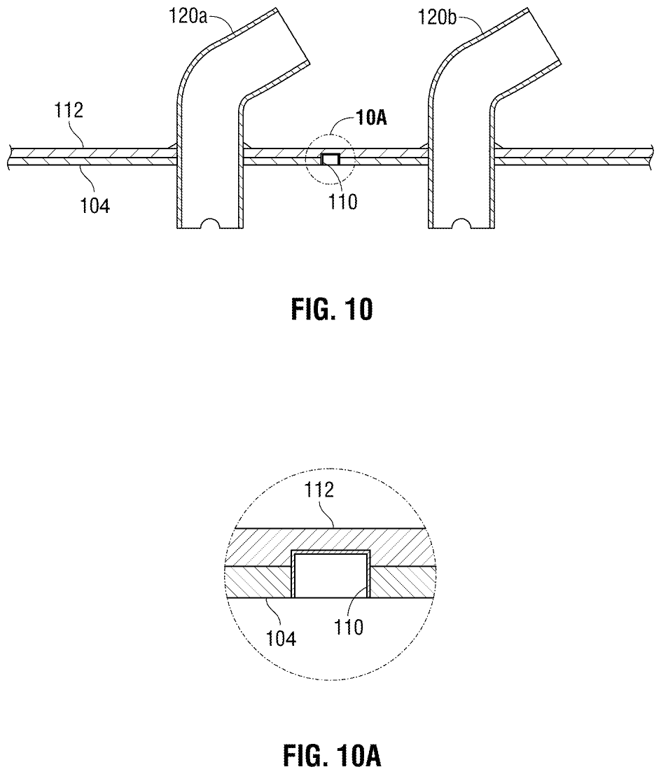

Referring now to FIGS. 1 to 10A, an agitation apparatus 100, which may be implemented as a vessel agitation apparatus, in accordance with one or more embodiments of the present disclosure may include: (1) an outer stainless-steel holding tub 102 as schematically depicted in FIGS. 1, 2 and 4; (2) a detachable and removable stainless-steel mesh basket or drum 104 (hereinafter, basket 104) as schematically depicted in FIG. 3; (3) an electronic clutch 106, an outer splined shaft 134, and an inner splined shaft or coupler 132 system in which the clutch 106 engages and disengages from the basket 104 as schematically depicted in FIG. 7; (4) a connection sealed bushing or bearing 110 which engages a shaft 137 of the disc 108 to a bottom portion of a cover or lid 112, which is illustrated in FIGS. 5, 10 and 10A, (5) a drive unit 114 including a DC motor 116 and pulley system 118 as schematically depicted in FIG. 6; (6) the hinged (e.g., stainless-steel) cover or lid 112 with solute and solvent supply connectors 120A, 120B as schematically depicted in FIG. 10; (7) a drain device 122 with a back pressure and filtration regulator (not shown) as schematically depicted in FIG. 8; and (8) a control panel 124 for automation and control of the agitation apparatus 100 as schematically depicted in FIGS. 1, 4, and 9.

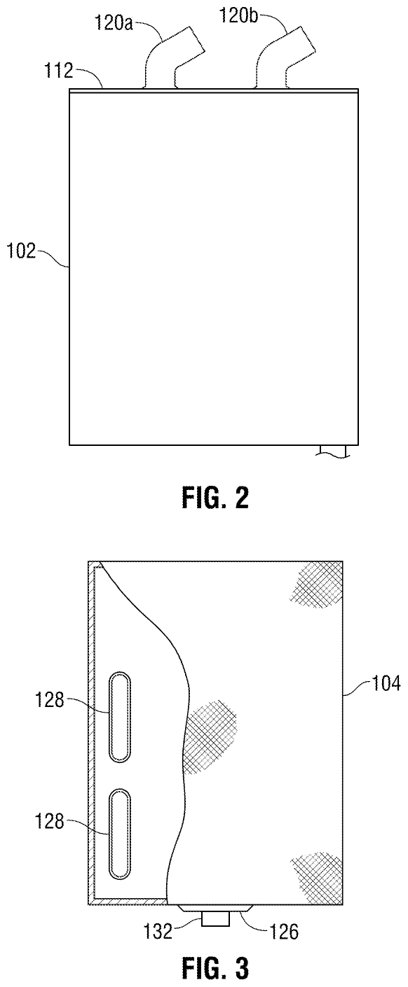

The agitation apparatus 100 shown in FIGS. 1 and 2 may be used to react hemp material with an organic solute in a water-based solution contained therein to extract carbon-containing fractions of cannabinoid acids from hemp material (e.g., CBDA, CBGA, CBCA, or the like) in an oxidized state. The automated agitation apparatus 100 may also be capable of effectively extracting polar cannabinoids as well as non-polar cannabinoids. In certain embodiments, the apparatus 100 may effectively extract the oxidized form of cannabinoids and the non-polar (i.e., hydrophobic) cannabinoids may only be extracted due to the agitation of the glandular trichomes. The ratio of acid to non-acid cannabinoids in the extraction may be similar to what the ratio is in the starting raw material. When plant material is fresher, the acid portion of ratio may be higher.

As discussed herein, these extractions may be obtained in the selective water-based weak alkaline solution from the hemp tissue. The agitation apparatus 100 may then be used to discharge the carbon-containing fractions of cannabinoids and the cannabinoid end products suspended within the weak alkaline solution from the agitation apparatus 100 by evacuation or draining through the drain device 122.

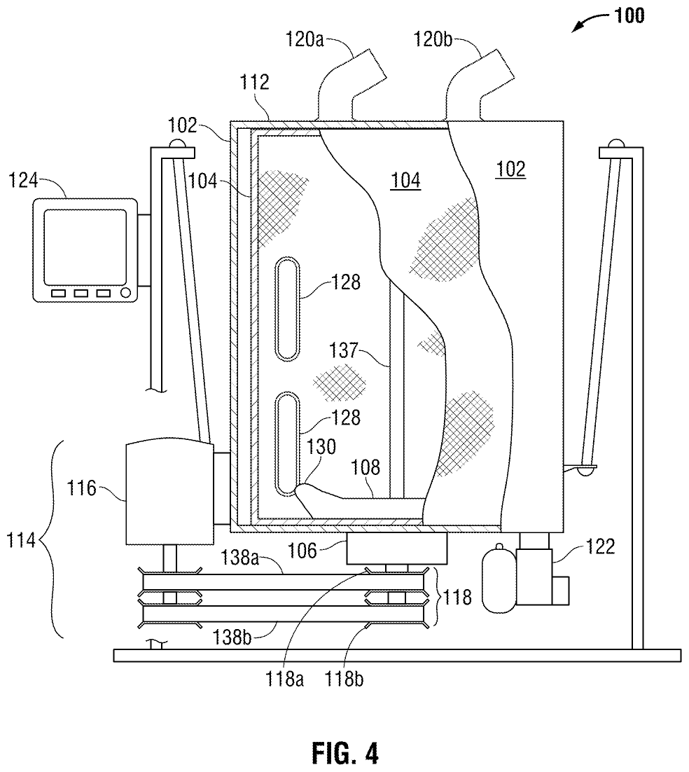

Before draining, the extracted polar and non-polar cannabinoids are filtered or screened through the food grade and detachable mesh screen basket 104 depicted in FIG. 3, as described herein, and into the interior portion of the larger diameter (e.g., stainless-steel) holding tub 102 depicted in FIGS. 1 and 2, that is, in between the basket 104 and the wall of the tub 102. FIG. 3 shows the mesh basket 104 of the agitation apparatus 100 of FIG. 1 may be reinforced with cage (e.g., stainless-steel) supports (not shown), a bottom plate 126 (e.g., stainless-steel), and turbulence nodes, bumps, lumps, notches, indentions, humps, or the like 128 as shown in FIGS. 3 and 4. The turbulence nodes 128 protrude inward toward the interior of the mesh basket 104 and extend in a vertical direction.

The agitation apparatus 100 may be capable of discharging large volumes of the cannabinoids in solution from the outer holding tub 102 via the drain system 122 depicted in FIG. 8. The agitation apparatus 100 also may be capable of blocking a backflow of solution via either a one-way floating valve or a backflow preventer device in the drain device 122 (e.g., as may be commonly incorporated in drain devices, although not shown in FIG. 8).

The agitation apparatus 100 may include the low-profile mixture disc or propeller 108, as depicted in FIG. 5, which is rotated by and coupled to the inner shaft 136 and thereby rotates, mixes, and agitates the hemp material and water-based solution within the inner mesh basket 104 (having specific micron-sized openings) for a specific amount of time by using the drive system 114. The drive system 114 includes (the motor 116, which may be a DC brushless motor, a lower pulley 118B of the pulley system 118 and a lower belt 138B as depicted in FIGS. 4 and 6. The drive system also includes an upper pulley 118A and an upper belt 138A for rotating the basket 104, The openings in the mesh basket 104 may be, for example, 70-125 microns inclusive. The mesh basket 104 (see FIG. 3) is secured into place via the short shaft extension 132 and into place via the outer splined shaft 134, as depicted in FIG. 7. The electronic clutch 106 mounted to the outer shaft 134, is similar to that disclosed in U.S. Pat. No. 8,235,196B2, which is incorporated herein in its entirety. The inner shaft 136 is mounted to the lower pulley 118B, which is directly driven via the V-belt 138B by the drive motor 116. The agitation disc 108 may be rotated to rotate, mix, and agitate the portion of the water-based solvent and weak alkaline solute of the solution enclosed within mesh or basket 104 inside the outer holding tub 102 during agitation time periods. After a specific agitation time period of, for example, selected in the range 30±10 minutes inclusive, the disc 108 may stop rotating and come to a complete stop. The electronic clutch 106 engages the outer shaft 134 (see FIG. 7), which mates to the mesh basket 104, allowing the mesh basket 104 to be fully rotated the drive motor 116 via the upper pulley 118A and the belt 138A (see FIG. 6). The high rotational speeds of the inner mesh basket 104 may impart centrifugal force on the hemp tissue to drive it against the inner wall of the mesh basket 104. The force, for example, may be in the range 150 G±25 G inclusive. This may further help any or some of the remaining polar and non-polar cannabinoids in the hemp tissue and solution to be extracted through the inner filtration mesh basket 104 and into the interior portion of the holding tub 102 between the mesh basket 104 and the wall of the tub 102.

The agitation disc 108 may include turbulence fins 130 and the shaft 137, as depicted in FIGS. 5 and 7. The drain device 122 depicted in FIG. 8 with an included ventilation valve (not shown) may connect to the outer tub 102, and inputs 120A, 120B for both solute and solvent, respectively, may be built into the stainless-steel lid 112 as depicted in FIGS. 1, 2, 4, and 10.

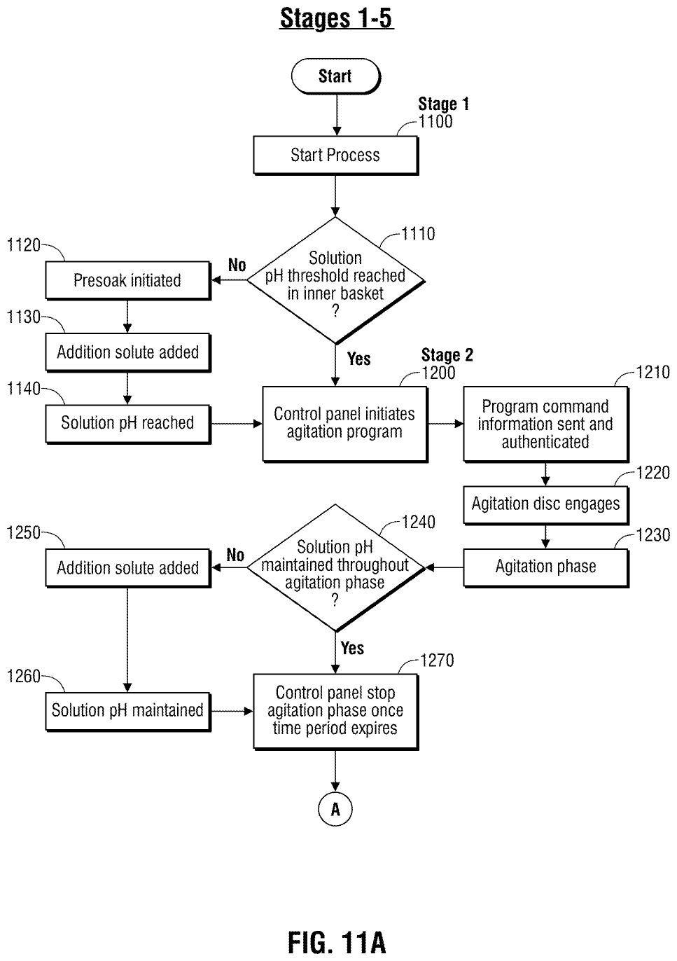

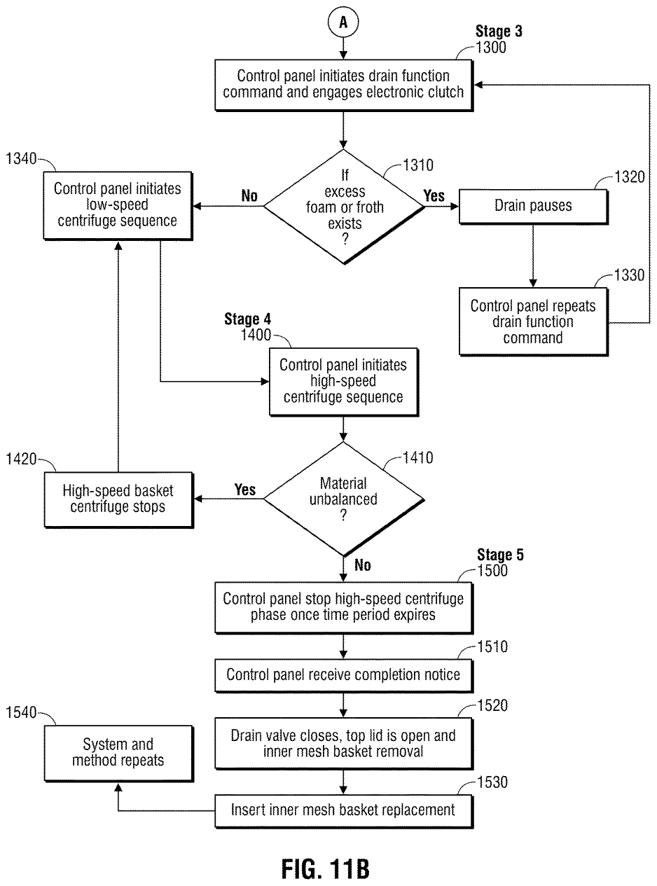

FIGS. 11A and 11B illustrate a flowchart of the operations of the system 100, apparatus, and methods described herein, in accordance with embodiments of the present disclosure. As illustrated in FIGS. 11A and 11B, the agitation apparatus 100 may provide a plurality of “stages” of operations. This plurality of stages may include multiple steps or processes for each stage, which may be repeated multiple times within each stage. In stage 1, the system 100 devices are powered on (start process) 1100 via the control panel 124 (see FIG. 9). A specific amount of filtered water solvent is added through the inlet 120B along with a weak alkaline solute in stage 1 via the inlet hose attachment 120A to the lid 112 of the agitation apparatus 100 until a specific solution pH is reached 1110. If the specific pH threshold is not reached, a presoak of the hemp tissue is initiated 1120 and additional weak alkaline solute to be added 1130 until the desired pH level is achieved 1140.

Referring again to stage 1 in more detail, presoaking the hemp tissue in solution 1120 helps rehydrate dry and semi dry flower and biomass, allowing the trichome glands to open up within the hemp tissue flower and/or biomass. The solution, as it fills the agitation apparatus 100, will pass through the openings in the mesh basket 104 and to fill the tub 102 as well until it levels out at a predetermined height. A soaking time of 10±5 minutes inclusive may then be used before any agitation 1200 in stage 2. Then or after a predetermined time, the control panel 124, automatically or under operator control, using a coded program, sends a signal to a driver board (not shown) that controls two separate steps or processes: (1) the electronic clutch 106 may be initialized to engage and disengage the outer shaft 134, which prevents the mesh basket 104 from moving and/or rotating.

Once the desired pH level is achieved, the control panel 124, using the coded program, then may send a signal to the driver board (not shown) to initiate activation of the driver motor 116 connected or mated via the pulley belt 138B to the pulley system 118 attached to the inner agitation shaft 136 for rotation of the disc 108 for agitation. Stage 2 initiates or launches the agitation program 1200 at the control panel 124. Program command information is sent from the control panel 124 and initiates 1210 multiple steps (program command information sent and authenticated) to control the motor 116 and the pulley 118B (see FIGS. 6 and 7), which is attached to the inner shaft 136 that is connected to the agitation disc 108. The agitation disc 108 is engaged 1220 and the shaft 136 rotates the agitation disc 108, which commences an agitation phase 1230 for a specific duration of time. The agitation disc 130 is connected to the inner agitation shaft 136, which rotates about its axis with periodic reversals of rotation direction, thus causing agitation of the hemp and solution, as will be described below. The disc 108 will rotate about the longitudinal axis of the agitation shaft 136. The inner agitation shaft 136 may be similar to that described in U.S. Pat. No. 8,235,196B2, which is incorporated by reference herein in its entirety. If the pH of the mixture in the solution is not maintained 1240 and drops below preferable limits during the extraction, the weak alkaline solute may be added 1250 to maintain a certain pH level 1260 until the agitation phase is completed when the time duration is reached or the time period expires 1270. For example, the weak alkaline solute may be added if the pH drops within after ten (10) minutes of agitation due to the weak alkaline solution continuously converting cannabinoid acids to their oxidized state within the solution.

In stage 3, as illustrated in FIG. 11B, under program control from the control panel 124, a drain function via the drain 122 (see FIG. 8) is initiated and the electronic clutch 106 (see FIG. 7) is engaged 1300. The electronic clutch 106 engages the outer shaft 134, which is connected to the mesh basket 104. If there is excess foam or froth 1310, the drain function will pause 1320 for a period of time, (e.g., 2-4 minutes) and will repeat 1330 under program control from the control panel 124 (i.e., at 1300), if necessary, until the foam or froth subsides. Then under program control, the control panel 124 initiates a low-speed centrifuge sequence 1340. The rotation of the mesh basket 104 is driven via the system 114 that includes the engaged electronic clutch 106 and an upper pulley 118A mounted to the drive motor 116 via the V-belt 138A (see FIG. 6).

In stage 4, under program control from the control panel 124, a high-speed centrifuge sequence is initiated 1400. If an unbalanced load of hemp tissue is sensed as the mesh basket 104 ramps up rotational speed 1410, then the high-speed basket centrifuging with the basket 104 will stop 1420. The program control from the control panel 124 will then reinitiate low speed centrifuging 1340 until the material load is balanced. If the load is balanced, the high-speed centrifuging 1400 with the mesh basket 104 will continue for a period of time (e.g., 7±3 minutes inclusive). Stage 4 may provide the dual functions of dissociating cannabinoid acids, cannabidiol, cannabinoid, terpenes, flavonoids, and chlorophyll from hemp tissue and suspending them within the weak alkaline solute and water solvent as it exits the mesh basket 104.

In stage 5, under program control from the control panel 124, the high-speed centrifuge phase is stopped once the time period has been reached 1500. The control panel 124 then receives or senses a completion notice or signal 1510, the drain valve in the drain 122 may be closed, the top lid 112 may be opened, and the inner mesh basket 104 may be removed 1520. Then a replacement inner mesh basket like the basket 104 may be inserted 1530. The system and methods described herein then may be repeated 1540, going back to the start of the process 1100 with a new batch of hemp material.

The lid 112 and lid portion shown in FIGS. 1, 2, 4, and 10 may provide the two inlet connections 120A (left) and 120B (right) for controlled inflow respectively, of solvent and solute (e.g., if the solute is in liquid form, although it may be solid form as well in certain embodiments of the present disclosure) into the mesh screen basket 104 using external and/or internal electronic and/or manual controlled valves (not shown) (e.g., for stages 1 and 2 described above). Flow lines may be provided for adding these materials (e.g., water, weak alkaline solute, etc.) into the agitation apparatus 100. A flow rate monitor (not shown) may be provided on both of the 120A and 120B inlets. Filtered neutral pH water (e.g., at 21° C. or at a temperature within the ranges described above) may be introduced through the inlet 120A at a flow rate of twenty-five (25) gallons per minute (95 liters/min) into the inner mesh basket 104 with a transfer or filling time period of about two (2) minutes. The secondary inlet 120B may be used to inject the weak alkaline solute into the water solvent to create weak alkaline solution having pH 10±2 inclusive. Any additional solvent and/or solute maybe introduced into the inner mesh basket 104 via the two inlets 120A, 120B.

With the inner mesh basket 104 locked in position as described above, the control panel 124, using the coded program, may then send a signal(s) to control the driver motor 116 to produce successive counter-directional rotations of the disc 108 (e.g., successive clockwise and counterclockwise rotations) to agitate the mixture (i.e., the hemp material in the solution). The rotational speed and direction of agitation disc 108 rotation may be continuously adjusted at a slow rate (at 1230), for example, for an initial 1±1 minute inclusive to help stabilize and balance hemp material evenly within the inner mesh basket 104 in solution as determined by sensors (not shown). Following stabilization of the hemp tissue in the mixture, the agitation disc 130 may be controlled by a signal(s) sent from the control panel 124, using the coded program, to rotate in alternating directional rotations for periods of time at a high rate(s) of speed. The disc 108 may be rotated in one of the directions (e.g., clockwise) for a period(s) of tune and then rotated in the other direction (e.g., counterclockwise) for another period(s) of time (which may be the same or different periods of time from each other). For example, these alternating periodic reversals in rotational motion may go on for approximately 15 seconds (e.g., 25 seconds±10 seconds inclusive) for each period of rotation.

In accordance with exemplary embodiments of the present disclosure, the following method or process may be used. Clockwise rotation of the disc may occur at 150-200 revolutions per minute (RPM) inclusive for a specified amount of time in seconds:
a. Control panel 124 is programed to send signals to rotate the disc for 15-35 seconds inclusive and a vortex of the mixture within the inner mesh basket 104 may be created.
b. Control panel 124 is programmed to send signals to pause rotation of the disc 108 for some time (e.g., 2-4 seconds inclusive) after a certain total number of revolutions has been achieved.
   Then counterclockwise rotation of the disc 108 may occur at 150-200 RPM inclusive for the same or a different specified amount of time in seconds compared to the clockwise rotation:
c. Control panel 124 is programed to send signals to rotate the disc 108 for 15-35 seconds inclusive and a vortex of the mixture within the inner mesh basket 104 may be created in the opposite direction from above.
d. Control panel 124 is programmed to send signals to pause rotation of the disc 108 for some time (e.g., 2-4 seconds inclusive) after the same as above or a different total number of revolutions has been achieved.
   This alternating sequence may be repeated and continued, for example, for 30 minutes (e.g., for stage 2, at 1230.

The inlet 120B may be used to dispense or provide additional weak alkaline solute into the mesh basket 104 and the tub 102 to maintain a specific pH of the mixture, which may be monitored by sensors (not shown) and displayed on a display on or coupled (e.g., wired or wirelessly) to the control panel 124. Furthermore, the supply inlet connection units 120A, 120B may be stably fixed and installed or connected to corresponding supply pipes, vessels, holding tanks, hoses, and/or feed lines (“supply pipes”) and such solvent (e.g., water) and weak alkaline solute may continue to be introduced into the two inlet connection units 120A, 120B as needed or desired to maintain pH or just to add the materials (e.g., stage 2, at 1250 and 1260).

It should be understood that the solution and cannabinoid components derived from the hemp continuously filter through the openings in the mesh basket 104 to the portion of the tub 112 between the basket 104 and the tub 102 while the coarse hemp tissue completely or substantially remains within the mesh basket 104.

Once the disc 108 agitation rotation is done within the given time frame, the control panel 124, under program control, may then send a signal to unlock the electromagnetic clutch 106, releasing the agitation disc 108. Another signal may then be sent by the control panel 124, under the coded program, which causes the mesh basket 104 to be locked via a locking mechanism (not shown) with the electronic clutch 106 engaged (see FIG. 7) (e.g., stage 3). If not disengaged, then the coded program control will pause the process. The control panel 124, under coded program control, may then send a signal to start rotational movement of the mesh basket 104 in a clockwise direction in an adjustable slow rotation (e.g., stage 3, at 1340), for example, for 60±30 seconds inclusive to stabilize the hemp tissue and solution mixture to equalize and level the mixture within the mesh basket 104. This rotational motion is termed “shake” in the coded program (e.g., stage 3, at 1340), a precursor to high-speed centrifuging. It is used to level out the hemp material inside the mesh basket 104 so when it starts the high-speed centrifuge process (e.g., stage 4, at 1400), the mixture is evenly distributed against the wall of the mesh basket 104. It primes the mixture for subsequent centrifugal extraction using the mesh basket 104 and draining of the desired cannabinoids and any remaining solution within the mesh basket 104 and hemp tissue.

The control panel 124, under coded program control, will send a signal to the drain device 122 of FIGS. 1, 4, and 8 to open a valve (not shown), for example, a solenoid valve, engage a backflow protection flap (not shown), and initiate an electrically driven drain pump rotor (not shown) inside the drain device 122. Draining the apparatus 100 may continue for a period of time, e.g., 2-3 minutes inclusive, or until the fluid (i.e., between the outside the mesh screen basket 104 and the wall of the tub 102) is drained. The draining device 122 may be paused (i.e., stopped from draining) while the valve remains open. This pause may be needed to allow any foaming or frothing of the mixture to subside as well as to allow material to settle in the mixture (e.g., stage 3, at 1310).

The control panel 124, under coded program control, then may send a signal to the drive motor 116 to rotate, causing the mesh basket 104 to rotate in a similar adjustable slow rotation and time period as described above, but in the opposite direction, that is, counterclockwise via the belt and pulley system 118. The control panel 124, under program control, may then reengage the drain device 122 to drain the fluid again during this slow rotation (e.g., stage 3, at 1340).

During stage 4, the rotational speed of the motor 116 may be gradually increased until the rotational speed of the mesh basket 104 reaches a certain RPM, e.g., 150 RPM, for a period of time, e.g., 1 minute. The rotational speed may then be increased over another period of time, e.g., 3 minutes, to reach a final RPM, e.g., 580-700 RPM inclusive or until 150 G-forces (±25 G-Force) is achieved. The particular RPM may scale with size and circumference of the mesh basket 104 and system. This rotational speed may be maintained for an additional period of time, e.g., 3 minutes. The control panel 124, under coded program control, may periodically shut off and close the valve (e.g., the solenoid valve) (not shown), a backflow flap (not shown), and the draining device 122 while shutting down the rotational spin if the hemp tissue is not evenly balanced and dispersed within the mesh basket 104 within the apparatus 100 (e.g., stage 4, at 1410). The coded program will reinitiate and repeat the drain and rotational function of the mesh basket 104 once the mesh basket is balanced. Once balanced, the drain device may then be used to effectively discharge the fluid containing the desired oxidized cannabinoids, terpenes, flavonoids, and chlorophyl to the outside and for collection (e.g., stage 5).

Once the basket has stopped rotating, the lid may be opened and the agitator disc 108 may be removed. The mesh basket 104 may then be removed via manual or electronic hoist out of the tub 102 and cleaned for reinsertion into position in the tub 102 or a replacement basket 104 may be moved into position in the tub 112 and then the agitator disc 108 may be repositioned in or with the mesh basket 104. At this point, the system, apparatus, and methods may be initialized again for repeating the processes for extraction from new material, as described herein.

The system, apparatus, and methods, in accordance with embodiments of the present disclosure, may provide multiple advantages for the hemp industry: for example, (1) cost, using such weak alkaline solute in filtered water, may be lower compared to the common methods using ethanol, hydrocarbon, or carbon dioxide and produce similar yields; (2) time for extraction with the agitation apparatus and the weak alkaline solute may be shortened (e.g., 10-20×), and (3) extraction may be performed at or near room temperature.

During operation of the system, apparatus, and methods described herein, various parameters may be specified, including batch size of hemp in kilograms (kg), total water consumption (filtered) in liters per kilogram (l/kg), transfer rate and amount of solution, and percent standing solution prior to the basket 104 centrifugal rotation. The time periods specified herein are estimated periods only and may be adjusted as needed or as determined after sampling acquired cannabinoid products during or while carrying out the methods or processes described herein or after analysis of the final extracted cannabinoids for desirability or specificity.

For each of the following parameters, using the system, apparatus, and methods described herein, exemplary minimum and maximum ranges of values are provided below, although variations of these parameters may be used instead, depending on the particular circumstances or by design or as determined by yield results. In some of the instances below, yields are discussed using the method without the usage of the apparatus 100. In these instances, solution and biomass are mixed in a vessel for producing chemical reactions only, and if the ethanol method of extraction is used, a different kind of mixing system is used compared to the agitation apparatus 100 disclosed herein.
a. The hemp tissue batch size (lbs.) may be between about 25 and 100 pounds (or 12 and 46 kilograms) inclusive.
b. The total filtered water consumption in gallons for the agitation apparatus 100 with dry to semi-dry flower and biomass hemp tissue (e.g., 5%-35% moisture content) may be between about 50 and 75 gallons (or 189 and 284 liters) inclusive.
c. The total filtered water consumption in gallons for the agitation apparatus 100 with semi dry to wet flower and biomass hemp tissue (e.g., 35%-50%+ moisture content) may be between about 30 and 50 gallons (or 113 and 189 liters) inclusive.
d. The percent (%) of solution amount with standard draining without centrifugal force may be about fifty percent (50%) compared to total initial solution amount. This means that without centrifuging using the inner mesh basket 104 and with just draining the solution alone, approximately 50% of the original amount of solution used will be drained and captured. The remaining solution may be stuck within the plant material inside the inner mesh basket 104. This is based on dry/semi-dry hemp stated in b above. If the hemp material is wetter (e.g., fresh biomass), then less solution and solute will be needed due to the increased volumetric amount of raw hemp tissue, which has greater water content.)
e. The percent (%) of solution amount with standard draining with centrifugal force may be 85%-92.5% inclusive compared to total initial solution amount.
f. Percentage yield of total cannabinoids, including all cannabidiols and their acid counterparts (in oxidized foul)), terpenes, flavonoids, and chlorophyll of extraction without using the methods, weak alkaline solute, and the agitation apparatus 100 described herein may be 1%-25% inclusive. This yield would be from just dispensing hemp tissue in a weak alkaline solution, letting it sit overnight, and then just draining the solution.
g. Percentage yield of total cannabinoids including all cannabidiols and their acid counterpart (in oxidized form), terpenes, flavonoids, and chlorophyll of extraction using the disclosed system, apparatus, and methods with weak alkaline solute may be no less 50%-90% inclusive.

It should be understood by one of ordinary skill in the art that the system and methods described herein may be scaled up by changing the size and/or type of the agitation apparatus 100 and the quantities and type of hemp tissue material, solute, solvent, and reactants. It also should be understood that, in the forgoing disclosure, temperatures given to the nearest degree may include all temperatures within a certain range (e.g., ±2.76° C. (±5° F.)) of a given value. While in a specific embodiment, a specific temperature or a temperature range may be given for various steps, such as condensation, humidity, operational temperature, or ambient room temperature, it should be clear to one of ordinary skill in the art that other temperatures outside these ranges may also be suitable. Also, in some embodiments, as disclosed, various components may be connected, mated, or coupled to each other, and other components may be added or may not be necessary, as would be understood by those of ordinary skill in the art. These may be direct or indirect connections or couplings. Further, the sequence of method steps or stages disclosed herein may be varied in their order or the steps or stages may be repeated, depending on desired outcomes or yields or as needed. Moreover, some steps or stages may be eliminated also depending on desired outcomes or yields or as needed.

It should be understood that the above-described embodiments are merely examples of possible implementations and that their description is not meant as a limitation because further embodiments, modifications and variations may be apparent or may suggest themselves to one of ordinary skill in the art. Many of such further embodiments, modifications and variations may be made without departing from the principles of the present disclosure. All such further embodiments, modifications and variations are intended to be included herein without departing from the scope of the following claims.

1. A method of extracting cannabinoids and cannabidiol acids in oxidized state from hemp tissue material (Cannabis sativa L.) in an agitation apparatus, comprising:
forming a mixture of hemp tissue material, comprising one or more of flowers, stem, leaves, or seeds, or a combination thereof, and a solution of an alkaline solute and water having a pH in a range of 10-12 inclusive;
extracting bioactive materials as one or more of cannabinoids and cannabinoid acids in oxidized form, terpenes, flavonoids, or chlorophyll from the hemp tissue material in the solution;
filtering the bioactive materials and portions of the solution; and
recovering the bioactive materials.

2. The method of claim 1, further comprising filtering the bioactive materials through a mesh screen basket having openings of any size in a range of 70-125 microns inclusive.

3. The method of claim 2, further comprising rotating the mesh screen basket, creating a centrifugal force on the bioactive materials.

4. The method of claim 1, further comprising releasing the bioactive molecules from glandular trichomes in the hemp tissue material during agitation.

5. The method of claim 1, wherein the subjecting the mixture to a reaction temperature comprises subjecting the mixture to a reaction temperature of 21° C. to form an oxidized crude cannabinoid extraction.

6. The method of claim 1, wherein the subjecting the mixture to a reaction temperature comprises subjecting the mixture to a reaction temperature of ≥21° C. to ≤26.5° C. to form an oxidized crude cannabinoid extraction.

7. The method of claim 1, wherein the subjecting the mixture to a reaction temperature comprises subjecting the mixture to a reaction temperature of ambient room temperature to form an oxidized crude cannabinoid extraction.

8. The method of claim 1, wherein the agitation apparatus comprises a single vessel agitation apparatus.

9. An agitation apparatus for extracting cannabinoids and cannabidiol acids in oxidated state from hemp tissue material (Cannabis sativa L.), comprising:
a holding tub;
a mesh screen basket configured to be removably disposed within the tub;
a clutch configured to removably lock the mesh screen basket;
a disc having fins and a shaft disposed within the mesh screen basket and configured to be agitatively driven by a motor, belt, and pulley system;
wherein the disc is configured to rotate clockwise and counterclockwise at certain speeds and torques for certain time periods and durations.

10. The apparatus of claim 5, wherein the mesh screen basket is configured to be reinforced with stainless steel cage supports having turbulence nodes for bumps, lumps, notches, indentions, humps, or the like.

11. The apparatus of claim 5, wherein the disc is configured to agitate hemp tissue in solution in the mesh screen basket.

12. The apparatus of claim 5, wherein rotation of the mesh screen basket is adjustable in speed, torque, and time period in both clockwise and counterclockwise directions.

13. The apparatus of claim 5 wherein the mesh screen basket is rotatable at a high rate of speed and provides centrifugal force to move bioactive molecules from hemp tissue material between the mesh screen basket and a wall of the holding tub.

14. The apparatus of claim 9, further comprising a draining device configured to attach to the holding tub to drain the bioactive molecules extracted from a solution filtered through the mesh screen basket.
